(19) United States
(12) Patent Application Publication
(10) Pub. No.: US 2019/0217156 A1
(43) Pub. Date: Jul. 18, 2019

(54) TRAINING SUPPORT SYSTEM AND TRAINING SUPPORT METHOD

(71) Applicant: CASIO COMPUTER CO., LTD., Tokyo (JP)

(72) Inventor: Satoshi MACHIDA, Tokyo (JP)

(73) Assignee: CASIO COMPUTER CO., LTD., Tokyo (JP)

(21) Appl. No.: 16/366,148

(22) Filed: Mar. 27, 2019

Related U.S. Application Data
(62) Division of application No. 15/353,176, filed on Nov. 16, 2016.

(30) Foreign Application Priority Data
Feb. 4, 2016 (JP) 2016-020210
Feb. 23, 2016 (JP) 2016-032112

(51) Int. Cl. A63B 24/00; A63B 71/06; G09B 19/00

(52) U.S. Cl. CPC A63B 24/0075; A63B 71/0622; A61B 5/02; G09B 19/0038; A63B 24/0062

(57) ABSTRACT

A training support system supports a user in performing physical training, wherein the physical training includes first training performed using a training machine and second training performed without using the training machine, the system consisting of a sensor attached on the user to obtain information of the user, a second-training information collecting unit for collecting from the sensor exercise information indicating an exercise volume of the second training, and a first-training menu notifying unit for preparing a menu of the first training reflecting the exercise information of the second training collected by the sensor and notifying the prepared menu of the first training to the user.

### CROSS-REFERENCE TO RELATED APPLICATION

This is a Divisional application of U.S. application Ser. No. 15/353,176, filed Nov. 16, 2016, which is based upon and claims the benefit of priority from the prior Japanese Patent Application No. 2016-020210, filed Feb. 4, 2016, and Application No. 2016-032112, filed Feb. 23, 2016, the entire contents of all of which are incorporated herein by reference.

### BACKGROUND OF THE INVENTION

### 1. Field of the Invention

The present invention relates to a training support system and training support method.

### 2. Description of the Related Art

In a training gym, a user performs training using a training machine such as a running machine. A method of measuring and/or managing the training by using the training machine is proposed, for example, in Japanese Unexamined Patent Publication No. 2012-170754 and Japanese Unexamined Patent Publication No. 2009-089799. The training is measured to prepare a training menu serving user's purpose of training. The user is allowed to perform training in accordance the training menu decided depending on the techniques disclosed in the above patent publications. As a result, the user will perform training effectively.

In recent, persons with various purposes of training come to the training gym, for instance, for the purpose of improving physical ability and for the purpose of maintaining health. Some persons could not come to the training gym so often due to some reason. These persons perform training (self training) at home in addition to performing training in the training gym, thereby finishing the scheduled training.

When the user performs training (self training) at home, a trainer of the training gym cannot accurately understand the content of the self training performed by the user. In other words, although the trainer of the training gym can understand the content of the self training roughly, it is hard for the trainer to prepare an effective training menu for the user. Therefor, the training actually performed by the user does not match the training menu prepared by the trainer, and it is hard to expect that the user performs training effectively.

### SUMMARY OF THE INVENTION

The present invention has been made to solve the problems residing in the conventional systems and provides a training support system and training support method which effectively supports a user in performing training.

According to one aspect of the present invention, there is provided a training support system for supporting a user in performing physical training, wherein the physical training includes first training performed using a training machine and second training performed without using the training machine, the system which comprises a sensor which is attached on the user to obtain information of the user, a second-training information collecting unit which collects from the sensor exercise information indicating an exercise volume of the second training, and a first-training menu notifying unit which prepares a menu of the first training reflecting the exercise information of the second training collected by the sensor and notifies the prepared menu of the first training to the user.

According to another aspect of the present invention, there is provided a training support system for supporting a user in performing physical training, wherein the physical training includes first training performed using a training machine and second training performed without using the training machine, and the training machine is provided with a sensor, the system which comprises a first-training information collecting unit which collects from the sensor of the training machine exercise information indicating an exercise volume of the first training, and a second-training menu notifying unit which prepares a menu of the second training reflecting the exercise information of the first training collected by the first-training information collecting unit and notifies the prepared menu of the second training to the user.

According to other aspect of the present invention, there is provided a training support method for supporting a user in performing physical training, wherein the physical training includes first training performed using a training machine and second training performed without using the training machine, and the user has a sensor attached on him or her, the method comprising the steps of a second-training information collecting step of collecting from the sensor exercise information indicating an exercise volume of the second training, and a first-training menu notifying step of preparing a menu of the first training reflecting the exercise information of the second training collected by the sensor and notifying the prepared menu of the first training to the user.

The training support system and training support method of the present invention will support the user in performing training effectively.

### BRIEF DESCRIPTION OF THE DRAWINGS

FIG. 1 is a view schematically showing a configuration of a training support system according to the embodiment of the invention.

FIG. 2 is a view schematically showing a configuration of a training support apparatus in the training support system according to the present embodiment of the invention.

FIG. 3A is a view showing an example of a data configuration of a self training information DB.

FIG. 3B is a view showing an example of a data configuration of a gym training information DB.

FIG. 4 is a sequence diagram showing a training menu preparing process performed by the training support system according to the embodiment of the invention.

FIG. 5 is a sequence diagram showing a self training support process performed by the training support system according to the embodiment of the invention.

FIG. 6 is a sequence diagram showing a gym training support process performed by the training support system according to the embodiment of the invention.

FIG. 7 is a view showing a self training menu displayed on a touch display device.

FIG. 8A is a view showing a today's gym training menu prepared in the case where self training was performed neither too much nor too little.

FIG. 8B is a view showing the today's gym training menu prepared in the case where the self training was performed insufficiently.

FIG. 8C is a view showing the today's gym training menu prepared in the case where that the self training was performed insufficiently but in consideration of the physical condition of a user.

FIG. 9A is a view showing a notice giving the alarm that a training volume has exceeded greatly more than set in the training menu.

FIG. 9B is a view showing a notice giving the alarm that the training cycle is too short.

FIG. 9C is a view showing a notice giving the alarm that the user has fell down.

### DETAILED DESCRIPTION OF THE PREFERRED EMBODIMENTS

The preferred embodiments of the invention will be described with reference to the accompanying drawings in detail. Each drawing illustrates the embodiment schematically to a degree such that the invention can be fully understood. Therefore, it will be understood that the invention is not limited only to the particular examples shown herein. In the drawings, like reference numerals or symbols designate like elements and a further description thereof will be omitted.

### Configuration of Training Support System

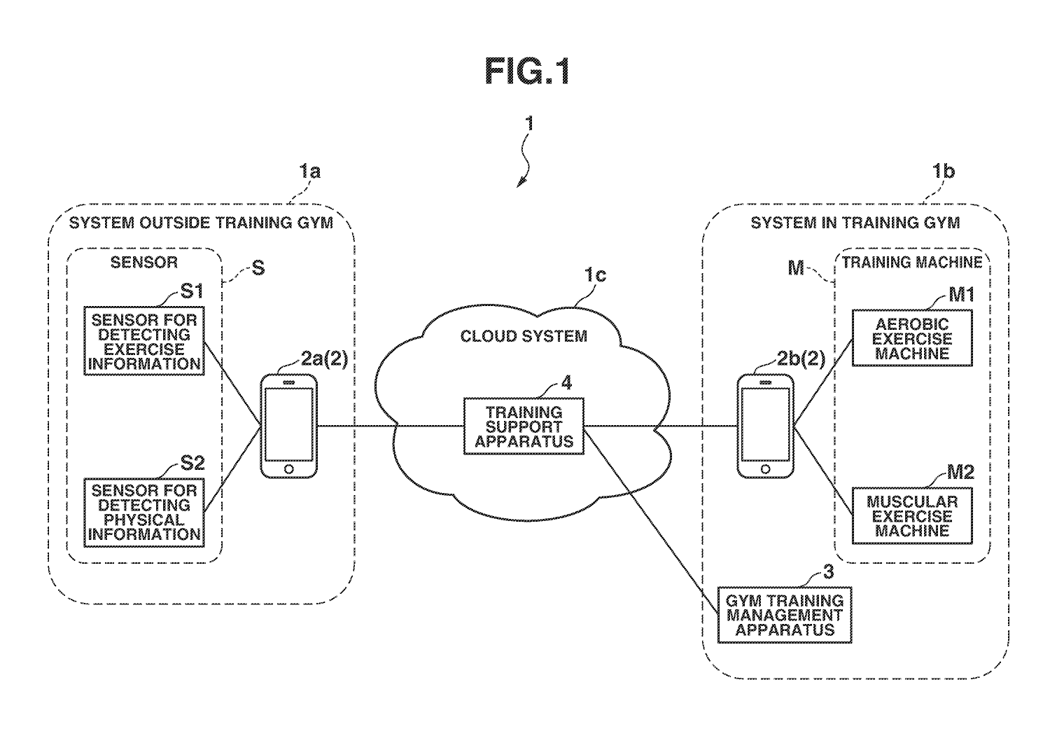

A training support system 1 according to the embodiment of the invention will be described with reference to FIG. 1. FIG. 1 is a view schematically showing a configuration of the training support system 1 according to the embodiment of the invention.

The training support system 1 provides users with an assisting service in a user's physical training. The training support system 1 can support any sort of training item as far as the training item has relation that can be previously anticipated, between a volume of performing training (for example, a training duration of time, a repetition frequency of training and so on) and a result of performing the training (for example, consumed calorie). Further, in the present embodiment of the invention, it is supposed that the training is performed in the training gym and also at places other than the training gym. The training gym is a facility which is used by users for executing training. The person who wants to improve physical ability and/or to maintain health visits the training gym to perform training. Hereinafter, the training performed in the training gym is particularly referred to as the “gym training”, and the training performed at a place other than the training gym is particularly referred to as the “self training”. The “gym training” is a “first training and the “self training” is a “second training”.

As shown in FIG. 1, the training support system 1 consists of a system la used outside the training gym, a system 1b used within the training gym, and a cloud system 1c which is capable of communicating with the system la outside the training gym and the system 1b in the training gym.

### System used Outside the Training Gym

The system 1a used outside the training gym is composed of a sensor S attached to the user and a user terminal 2a used by the user.

Any sensor can be used as the sensor S, as far as such sensor is capable of detecting various sorts of information of the user. In FIG. 1, two sensors S1 and S2 are illustrated, that is, an exercise information detecting sensor S1 for detecting information (exercise information) of exercise performed by the user and a physical information detecting sensor S2 for detecting physical information of the user are shown. The exercise information detecting sensor S1 detects the exercise information such as consumed calorie and fat combustion quantity. The physical information detecting sensor S2 detects the physical information such as a heart rate, blood pressure, sleeping hours, and sleeping quality of the user. The sensor S has a function of a short distance communication and sends the user terminal 2a the information detected from the user.

The user terminal 2a is provided with an information processing function, input/output function, and communication function. For example, a multi-function cellular phone (smart phone) can be used as the user terminal 2a. The user terminal 2a has a training support application installed therein. When the application is started, the user can receive the training support from the cloud system 1c through the user terminal 2a. When the training support application is started, the user terminal 2a downloads a menu of the self training from the cloud system 1c to display said menu on a touch-display device. Further, the user terminal 2a starts communication with the sensor S to send the cloud system lc the information detected by the senor S.

### System used within the Training Gym

The system 1b used within the Training Gym is composed of a training machine M which is used by the user to perform training, the user terminal 2a used by the user, and a gym training management apparatus 3 owned by a company operating the training gym.

The training machine M is an apparatus which is used by the user to execute training, and is not restricted to a specific sort of apparatuses. In FIG. 1, two sorts of machines M1, M2 are illustrated, that is, an aerobic exercise machine M1 and a muscular exercise machine M2 are shown. The aerobic exercise machine M1 is used for performing an aerobic exercise and includes, for example, a running machine and aero-bicycle. The muscular exercise machine M2 is used to strengthen the muscles and includes, for example, a chest press and leg press. The training machine M has a built-in sensor for detecting various sorts of information of the user and the training conditions, and sends the user terminal 2b the information and training result detected by the built-in sensor. The training machine M can be provided with plural number of sensors.

The user terminal 2b is provided with an information processing function, input/output function, and communication function. For example, the multi-function cellular phone (smart phone) can be used as the user terminal 2b. It is possible to use the user terminal 2a as the user terminal 2b. Although the user terminal 2a and the user terminal 2b are shown in FIG. 1, the user is allowed to use one terminal as the two terminals (the user terminal 2a and the user terminal 2b). Hereinafter, when it is not required to designate the user terminal 2a and the user terminal 2b separately, sometimes the user terminal will be simply referred to as the “user terminal 2”. When the training support application is started, the user terminal 2b downloads a menu of the gym training from the cloud system lc to display said menu on the touch-display device. Further, the user terminal 2a starts communication with the training machine M to send the cloud system 1c the training state and result obtained from the training machine M. Meanwhile, it is possible for the company operating the training gym to rent the user terminal 2b to the user doing exercise in the gym. In this case, the user terminal 2a is a separate one from the user terminal 2b.

The gym training management apparatus 3 is provided with an information processing function, input/output function, and communication function and is used to manage the gym training. For example, a stationary personal computer equipped with a reading device is used as the gym training management apparatus 3. The gym training management apparatus 3 has a gym training management application installed therein. When the application is started, the gym training of the user can be managed on the gym training management apparatus 3. When the gym training management application is started, the gym training management apparatus 3 can watch the user having the user terminal 2 who enters and/or leaves the gym to perform access management of the user, and sends the cloud system 1c the gym access information of the user who enters and/or leaves the training gym. The gym training management apparatus 3 assists the trainer belonging the training gym in preparing the training menus (gym training menu and self training menu) for the user. The prepared training menus are stored in the cloud system 1c and the user is allowed to download the menus through his or her user terminal 2.

### Cloud System

The cloud system 1c is provided with a training support apparatus 4. The training support apparatus 4 is, for example, an application server which provides assisting service to the user in performing physical training exercise. The training support apparatus 4 can be composed of a single device and also plural devices. The training support apparatus 4 is provided on the Internet and can communicate with the user terminal 2 and the gym training management apparatus 3.

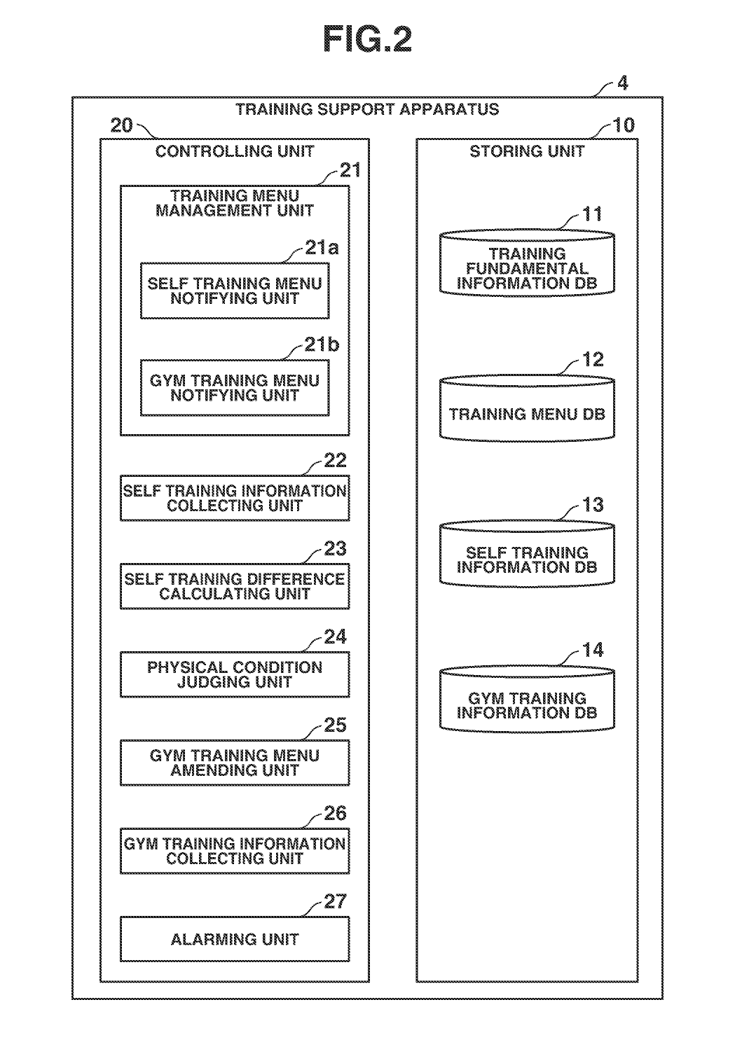

The training support apparatus 4 will be described with reference to FIG. 2. FIG. 2 is a view schematically showing a configuration of the training support apparatus 4 in the training support system 1 according to the present embodiment of the invention.

The training support apparatus 4 is provided with a storing unit 10 and a controlling unit 20. The storing unit 10 is composed of storing media such as RAM (Random Access Memory), ROM (Read Only Memory), HDD (Hard Disk Drive) and Flash memory. The controlling unit 20 is realized by special circuits and/or a processing program executed by CPU (Central Processing Unit).

When the controlling unit 20 is realized by the processing program executed by CPU, the processing program (a training support program) for realizing the controlling unit 20 is stored in the storing unit 10.

The storing unit 10 has a training fundamental information database (DB) 11, training menu database (DB) 12, self training information database (DB) 13 and gym training information database (DB) 14.

The training fundamental information DB 11 stores various sorts of information concerning the training. Volumes of consumed calorie per unit time and/or per unit cycle of training respectively corresponding to various sorts of gym trainings are stored in the training fundamental information DB 11.

The training menu DB 12 stores the training menus for the users. Each training menu includes the gym training menu and the self training menu. In the present embodiment, the training menu is prepared by the trainer using the gym training management apparatus 3 but the training menu which is created by the user himself or herself or by other person may be used. The training menu is decided, for example, based on the calorie (target consumed calorie) that the user has to consume in a day. The target calorie to be consumed in a day can be calculated, for example, from the following equation (1).

The target consumed calorie per day (kcal)=(somatic fat volume to be reduced (g)/days required for reducing the fat)×(calorie/somatic fat 1 g (kcal)) (1)

In the present embodiment, it is supposed that the user is a woman of 40 years old and she wants to reduce her somatic fat volume by 3 kg within two months (60 days).

Now, her training target will be described. Assuming that the somatic fat 1 g is about 7.2 kcal, it is required to consume the calorie “7.2 kcal×3000 g=21600 kcal” in toto to reduce the somatic fat by 3 kg. Since the training period is 60 days, the target consumed calorie per day will be “21600 kcal/60 days=360 kcal/day”. To consume 360 kcal, 60 minutes are required, if the training is walking and 30 minutes are required, if the training is running. Accordingly, the one-day menu of the gym training for the user will be “running machine: 30 minutes” and the one-day menu of the self training will be “walking: 60 minutes”.

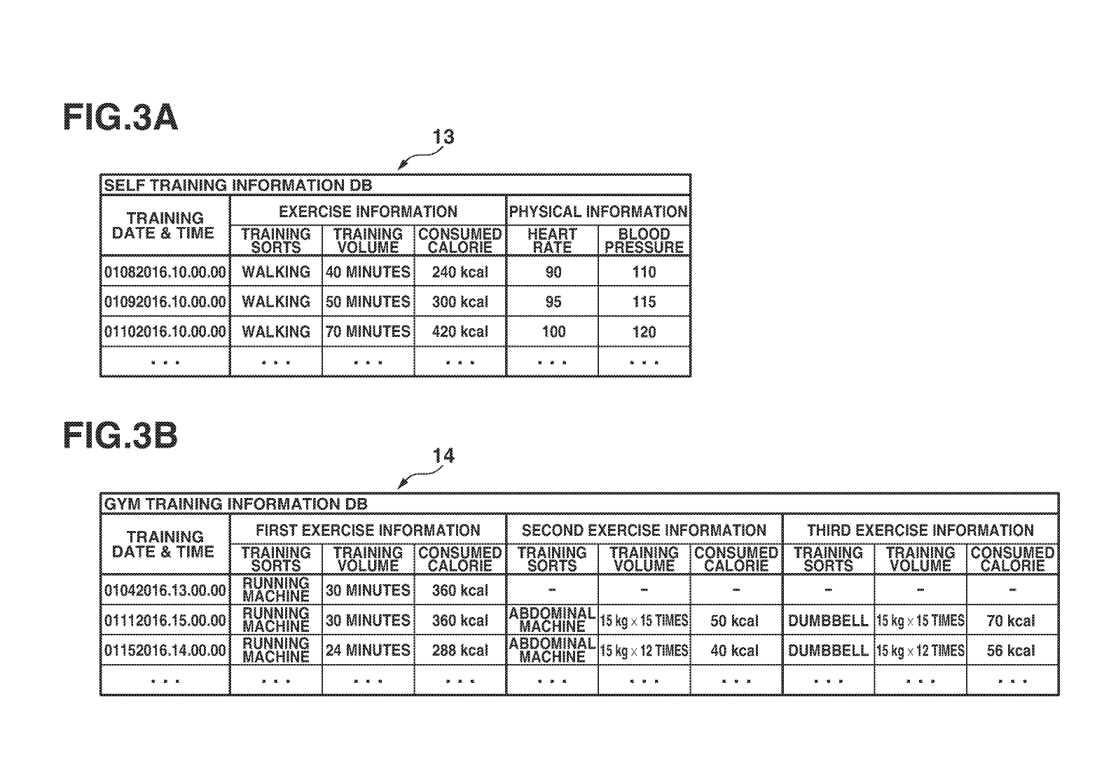

The self training DB 13 stores information (self training information) of the self trainings for the users. The self training DB 13 is updated based on the information sent from the user terminal 2a every time the user performs self training. The self training information is composed of, for example, exercise information and physical information associated with training dates. An example of a data configuration of the self training information DB 13 is shown in FIG. 3A.

In FIG. 3A, training dates and times indicate dates and times on which the user performed self training (for example, training starting dates and times, and training finishing dates and times). The exercise information is information concerning the exercise of the self training. The exercise information includes a training sort, training volume and consumed calorie. The training sort is information for designating the sort of the training. For example, the training sort indicates the name of the training. The training volume indicates the number of times or a time duration in which the user performs training. The consumed calorie indicates a value obtained from the product of the consumed calorie in unit time or in unit cycle of each training and the number of times or time duration in which the user performs training. The physical information is information concerning the body of the user and includes heart rates and blood pressures.

The gym training DB 14 stores information (gym training information) of the gym trainings for the users. The gym training DB 14 is updated based on the information sent from the user terminal 2b every time the user performs gym training. The gym training information is composed of, for example, exercise information associated with training dates. An example of a data configuration of the gym training information DB 14 is shown in FIG. 3B.

In FIG. 3B, the training dates and times indicate dates and times on which the user performed gym training (for example, training starting dates and times, and training finishing dates and times). The exercise information is information concerning the exercise of the gym training. The gym training DB 14 stores first exercise information, second exercise information, and third exercise information, depending on the training sorts. Each piece of exercise information includes the training sort, training volume and consumed calorie. The training sort is information for designating the sort of the training. For example, the training sort indicates the name of the training. The training volume indicates the number of times or a time duration in which the user performs training. The consumed calorie indicates a value obtained from the product of the consumed calorie in unit time or in unit cycle of each training and the number of times or time duration in which the user performs training.

Returning to the configuration of the training support apparatus 4 shown in FIG. 2, the controlling unit 20 will be described. The controlling unit 20 comprises a training menu management unit 21, self training information collecting unit 22, self training difference calculating unit 23, physical condition judging unit 24, gym training menu amending unit 25, gym training information collecting unit 26, and alarming unit 27.

The training menu management unit 21 manages the training menu. The training menu management unit 21 comprises a self training menu notifying unit 21a and gym training menu notifying unit 21b.

The self training menu notifying unit 21a (second training menu notifying unit) notifies the self training menu stored in the training menu DB 12 to the user terminal 2a which has sent a request for the training menu. Receiving the self training menu from the self training menu notifying unit 21a, the user terminal 2a displays the received self training menu on the touch display device, whereby the user is allowed to perform self training in accordance with the self training menu displayed on the touch display device.

The gym training menu notifying unit 21b (first training menu notifying unit) notifies the gym training menu stored in the training menu DB 12 to the user terminal 2b which has sent the request for the training menu. Receiving the gym training menu from the gym training menu notifying unit 21b, the user terminal 2b displays the received gym training menu on the touch display device, whereby the user is allowed to perform gym training in accordance with the gym training menu displayed on the touch display device. The gym training menu notifying unit 21b notifies the user terminal 2b of a gym training menu reflecting an excess and/or deficiency in the self training actually executed by the user to the self training menu notified by the self training menu notifying unit 21a or a difference between them. The detail thereof will be described later.

The self training information collecting unit 22 (second training information collecting unit) collects exercise information and physical information detected by the sensor S attached on the user and stores the collected information in the self training information DB 13.

The self training difference calculating unit 23 calculates an excess and/or deficiency in the exercise information stored in the self training information DB 13 to the self training menu stored in the training menu DB 12 or a difference between them. For example, as described above, assuming that the target consumed calorie per day is “360 kcal/day”, and the total calorie actually consumed during the period from “Jan. 8, 2016 to Jan. 10, 2016” is “240 kcal+300 kcal+420 Kcal=960 kcal”, then the calorie consumed in three days is less than the target consumed calorie by 120 kcal. The self training difference calculating unit 23 calculates this difference (=120 kcal). The case where the consumed calorie is less than the target consumed calorie has been described here, but the self training difference calculating unit 23 calculates the difference in the opposite case (excess training).

The physical condition judging unit 24 judges the physical condition of the user based on the physical information stored in the self training information DB 13. For example, the physical condition judging unit 24 calculates averages of the heart rates and/or blood pressures in a predetermined period of time stored in the self training information DB 13 and judges whether the last heart rate and/or blood pressure fall within the allowable ranges of the calculated heart rate and/or blood pressure averages. A method of judging the physical condition is not restricted to the described above, but it is possible to determine the physical condition depending on judging whether the last heart rate and/or blood pressure exceed predetermined threshold values of the heart rate and/or blood pressure.

The gym training menu amending unit 25 reflects the difference calculated by the self training difference calculating unit 23 and the physical condition determined by the physical condition judging unit 24 on the gym training menu stored in the training menu DB 12 to prepare a new gym training menu.

For example, when the consumed calorie is less than the target consumed calorie, the gym training menu amending unit 25 increases a training volume or adds an additional training sort in the new gym training menu to compensate the same. On the contrary, when the calorie is consumed more than the target consumed calorie, the gym training menu amending unit 25 decreases a training volume or a training sort in the new gym training menu to compensate the same.

Further, when the heat rate in the physical condition of the user is on the high side, the gym training menu amending unit 25 decreases the training volume by weighting. When the heat rate is on the high side, the gym training menu amending unit 25 weights the training volume by 0.8.

The gym training information collecting unit 26 collects various sorts of information of the user and training states performed by the user from the training machine M provided in the training gym. The information collected by the gym training information collecting unit 26 includes information concerning the training volume, information concerning the training cycle, and information concerning the training state.

For instance, when the user has performed training more excessively than the predetermined threshold volume, the information concerning the training volume indicates the fact. When the user has performed training in a shorter period than the predetermined cycle, then the information concerning the training cycle indicates the fact. When a trouble should happen to the user while performing training, the information concerning the training state indicates the trouble.

Further, the gym training information collecting unit 26 collects results of the training performance from the training machines in the training gym and stores the results in the gym training information DB 14.

The alarming unit 27 gives the alarm to the user terminal 2b based on the information collected by the gym training information collecting unit 26. It is preferable for the gym training information collecting unit 26 to give the alarm immediately after the gym training information collecting unit 26 has collected the information of the information, but the time preferable for giving such alarm is not restricted to such timing. Further, it is possible to accumulate information collected by the gym training information collecting unit 26 and give the alarm at the time when the user performs the following training.

### Operation of Training Support System

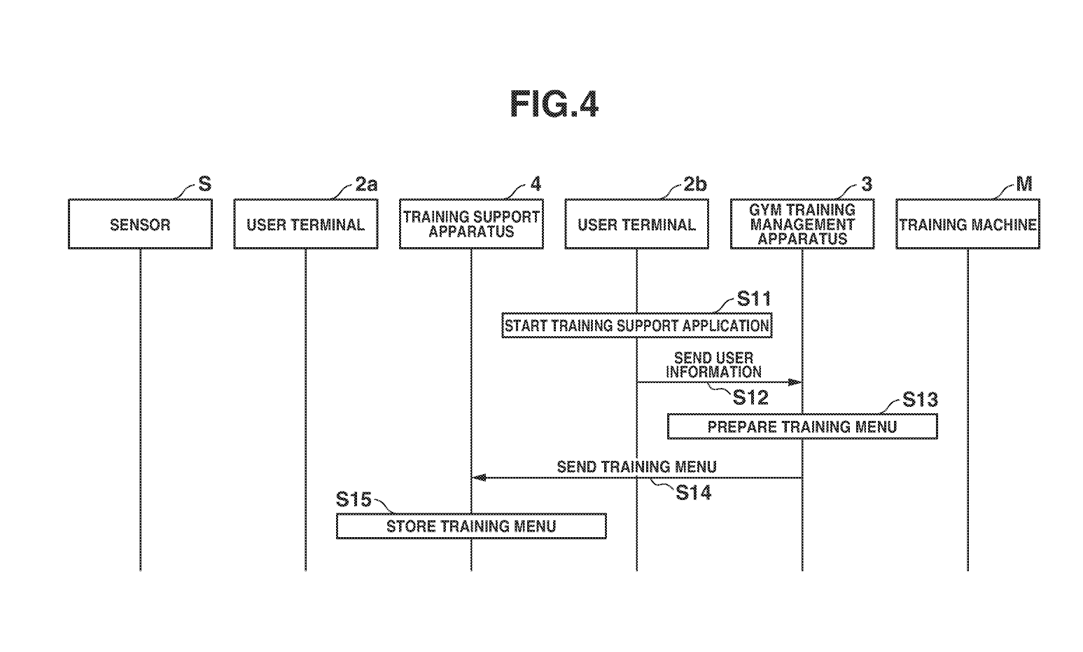

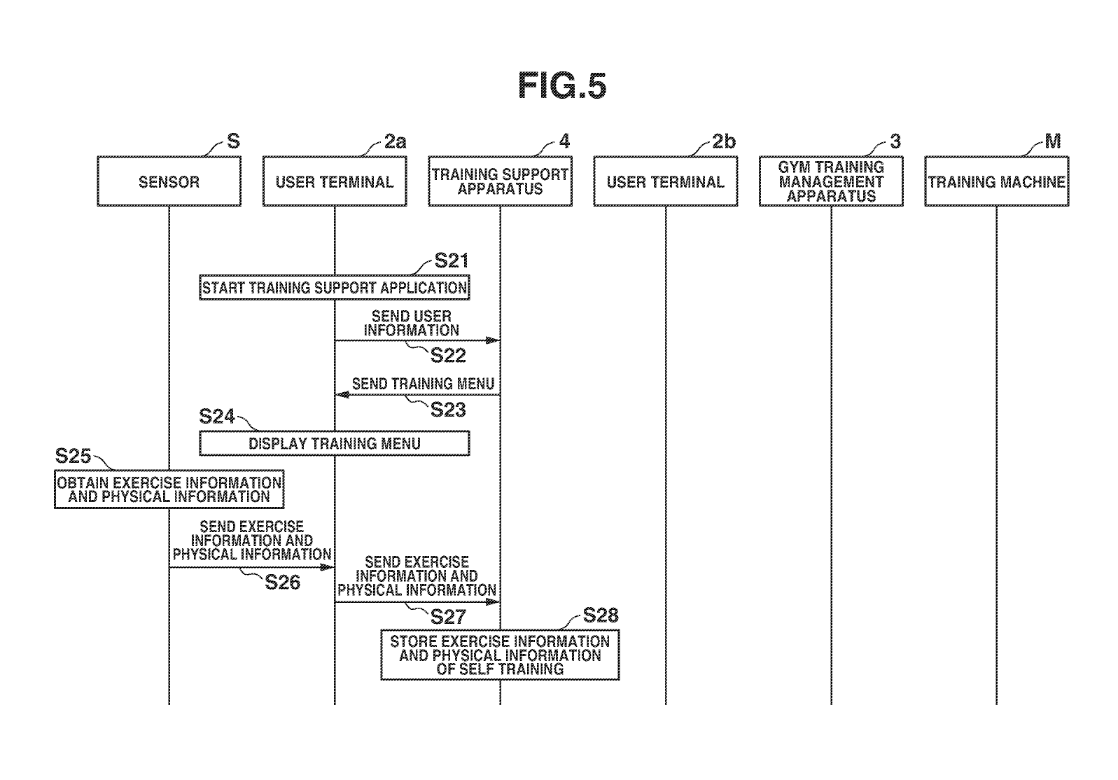

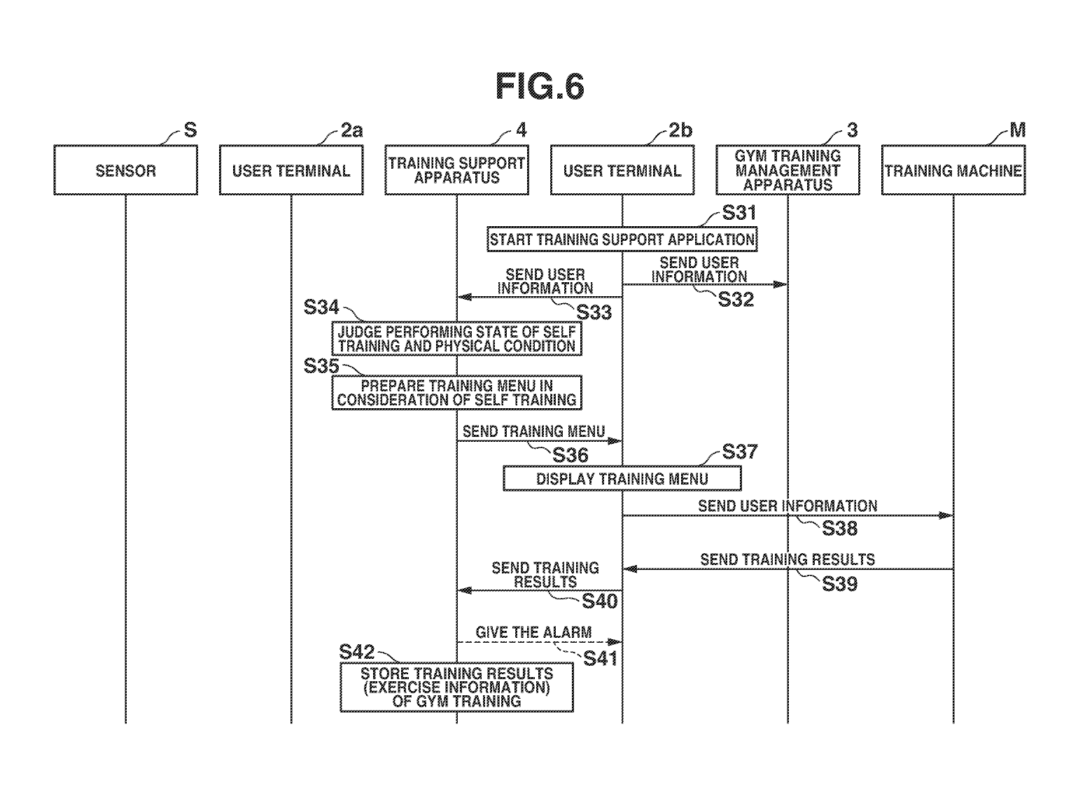

The operation of the training support system 1 according to the embodiment of the invention will be described with reference to FIG. 4, FIG. 5, and FIG. 6. The operation of the training support system 1 according to the embodiment of the invention mainly comprises a “training menu preparing process”, “self training support process”, and “gym training support process”.

FIG. 4 is a sequence diagram showing the training menu preparing process performed by the training support system 1 according to the embodiment of the invention.

FIG. 5 is a sequence diagram showing the self training support process performed by the training support system 1 according to the embodiment of the invention.

FIG. 6 is a sequence diagram showing the gym training support process performed by the training support system 1 according to the embodiment of the invention. Hereinafter, these processes will be described separately.

### Training Menu Preparing Process

The training menu preparing process will be described with reference to FIG. 4. The user comes to the training gym, and starts the training support application of the user terminal 2b (step S11). Then, the user holds the user terminal 2b up to the gym training management apparatus 3, whereby the user information stored in the user terminal 2b is sent to the gym training management apparatus 3 (step S12) and the user is permitted to enter the training gym. The trainer counsels or consults with the user and prepares a training menu using the gym training management apparatus 3 (step S13).

The gym training management apparatus 3 associates the prepared training menu with the user and sends said training menu to the training support apparatus 4 (step S14). The training menu management unit 21 of the training support apparatus 4 stores the received training menu in the training menu DB 12 (step S15).

### Self Training Support Process

The self training support process will be described with reference to FIG. 5. The user starts the training support application of the user terminal 2a (step S21), and enters a start of self training, whereby the user terminal 2a sends the user information to the training support apparatus 4 (step S22), and the self training menu notifying unit 21a of the training support apparatus 4 sends back the self training menu stored in the training menu DB 12 to the user terminal 2a (step S23).

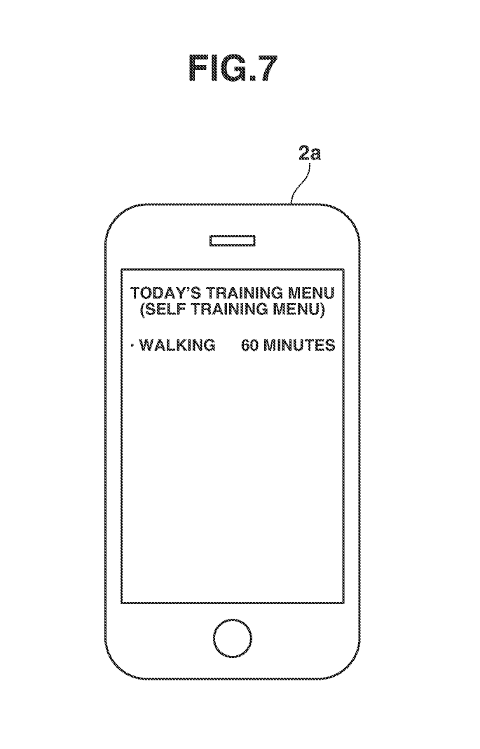

Receiving the self training menu from the self training menu notifying unit 21a, the user terminal 2a displays the received self training menu on the touch display device (step S24). The self training menu displayed on the touch display device at step S24 is shown in FIG. 7.

Then the user performs self training in accordance with the self training menu displayed on the user terminal 2a. The sensor S attached on the user obtains the exercise information and physical information of the user (step S25), and sends the obtained exercise information and physical information to the user terminal 2a (step S26). Further, the user terminal 2a sends the training support apparatus 4 the exercise information and physical information received from the sensor S (step S27). It is possible for the user terminal 2a to send the exercise information and physical information collectively, when the user finishes the training.

The self training information collecting unit 22 of the training support apparatus 4 stores the exercise information and physical information received from the user terminal 2a in the self training information DB 13 (step S28).

### Gym Training Support Process

The gym training support process will be described with reference to FIG. 6. The user comes to the training gym, and starts the training support application of the user terminal 2b (step S31). Then, the user holds the user terminal 2b up to the gym training management apparatus 3, whereby the user information stored in the user terminal 2b is transferred to the gym training management apparatus 3 (step S32) and the user is permitted to enter the training gym. Further, the user operates the touch display of the user terminal 2b to enter the start of the gym training, whereby the user terminal 2b sends the user information to the training support apparatus 4 (step S33).

The self training difference calculating unit 23 of the training support apparatus 4 calculates a difference between the self training actually performed by the user and the self training menu, and the physical condition judging unit 24 judges the physical condition of the user (step S34).

Further, the gym training menu amending unit 25 reflects the difference calculated by the self training difference calculating unit 23 and the physical condition determined by the physical condition judging unit 24 on the gym training menu stored in the training menu DB 12 to create a new gym training menu (step S35).

The gym training menu notifying unit 21b notifies the new gym training menu created at step S35 to the user terminal 2b (step S36), and the user terminal 2b displays the new gym training menu on the touch display device (step S37).

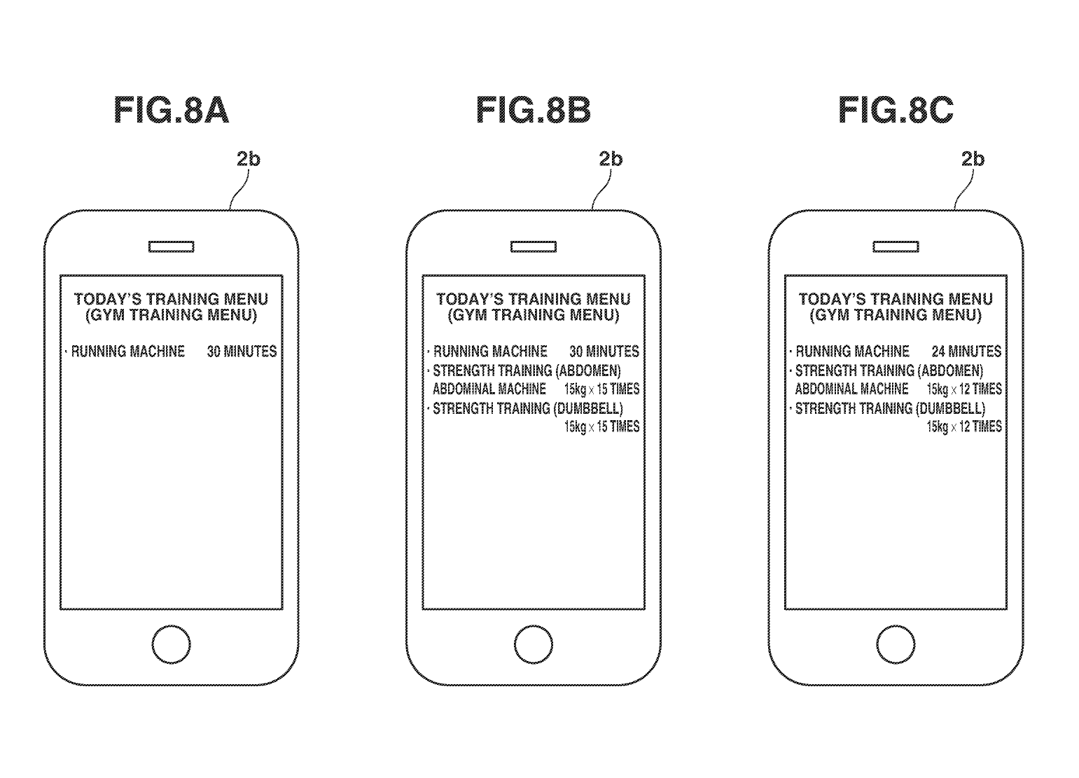

The new gym training menu displayed on the touch display device at step S37 is shown in FIG. 8A to FIG. 8C. FIG. 8A is a view showing the today's gym training menu prepared in the case where the self training was performed neither too much nor too little. FIG. 8B is a view showing the today's gym training menu prepared in the case where the self training was performed insufficiently. FIG. 8C is a view showing the today's gym training menu prepared in the case where that the self training was performed insufficiently but in consideration of the physical condition of the user.

In the gym training menu of FIG. 8B, strength training is added to the training menu of FIG. 8A. In gym training menu of FIG. 8C, the training volume is reduced compared with the training menu of FIG. 8A.

Returning to the sequence diagram of FIG. 6, the following process will be described. The user performs gym training in accordance with the gym training menu displayed on the user terminal 2b. The user holds the user terminal 2b up to the reading device of the training machine M or uses a communication circuit to send the user information to the training machine M (step S38), whereby the training machine is set in conformity with the gym training menu. The training machine M sends back the training results (for example, the exercise information) to the user terminal 2b (step S39). Further, the user terminal 2b sends the training support apparatus 4 the training results received from the training machine M (step S40). It is possible for the user terminal 2b to send the training results collectively when the user finished the training.

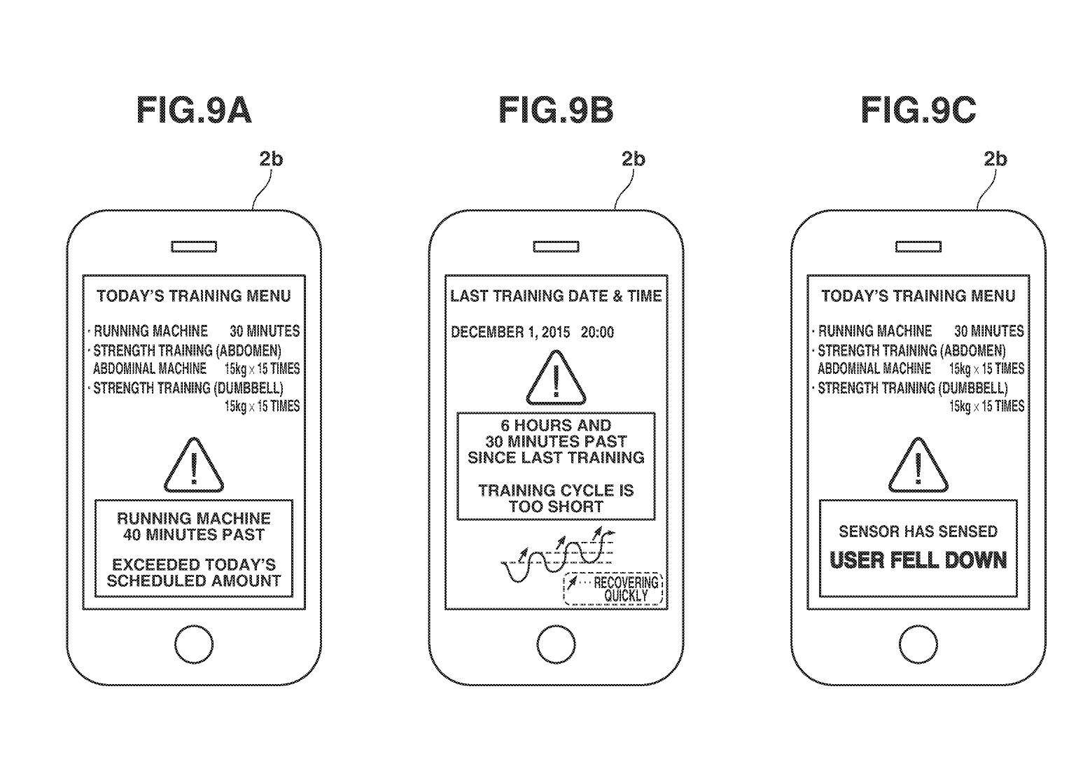

The alarming unit 27 of the training support apparatus 4 gives the user terminal 2b an appropriate alarm based on the information received from the user terminal 2b (step S41). Examples of the alarm displayed on the user terminal 2b at step S41 are shown in FIG. 9A, FIG. 9B, and FIG. 9C. FIG. 9A is a view showing a notice giving the alarm that the training volume has exceeded greatly more than set in the training menu. FIG. 9B is a view showing a notice giving the alarm that the training cycle is too short. FIG. 9C is a view showing a notice giving the alarm that the user has fell down. The gym training information collecting unit 26 stores the training results received from the user terminal 2b in the gym training information DB 14 (step S42).

As described above, the training support system 1 according to the embodiment of the invention calculates the excess and/or deficiency in the self training to the self training menu or the difference between them and reflects the calculated difference to the gym training menu. Therefore, even though there is the excess and/or deficiency in the training volume performed by the user to the previously determined dairy training menu, the target consumed calorie can be consumed accurately in toto.

The training support system 1 according to the embodiment of the invention judges the physical condition of the user while he or she is performing training, and reflects the judgment result to the gym training menu. Therefore, even when the user is not good in the physical condition, since his or her training volume is restricted, an accident due to the poor physical condition of the user will be avoided.

The training support system 1 according to the embodiment of the invention obtains the information concerning the training volume, information concerning the training cycle, and information concerning the training state from the training machine M, while the user is performing training, and gives the alarm to the user through the user terminal 2. Therefore, it can prevent the user from performing training excessively, and with being injured by the training machine M and/or being involved in trouble during performing training.

### Modification

The embodiments of the invention have been described hereinabove, but the invention is not limited to the particular embodiments described herein, and it may work the invention in modified and rearranged manners while remaining within the scope of the invention as defined by the following claims.

In the above embodiments of the invention, it has been supposed that the user performs training in the training gym and outside the training gym. And the gym training performed using the training machine M in the training gym is defined as the “first training”, and the self training performed without using the training machine M outside the training gym is defined as the “second training”. But the training performed by the user will not be restricted to these first and second trainings.

For instance, when it is assumed that the training machine M is busy and the user performs training while he or she is waiting for his or her turn (waiting period), it is possible to define the training (machine training) performed using the training machine M as the “first training”, and the training (non machine training) performed without using the training machine M in the waiting period as the “second training”. It is preferable to notify the user of a machine training menu reflecting the information collected during the non machine training by the sensor S.

The training using the training machine M (machine training) and the training performed without using the training machine M (non machine training) will not always be performed in the training gym but also outside the training gym.

It will be preferable to place importance on a training environment, too. It is possible to define the training performed in a good environment (first environment) maintained in a good condition for training as the “first training” and the training performed in a poor environment (second environment) maintained in a poor condition for training as the “second training”. It is possible to include various elements such as the training machine M, a training coach, an air conditioning machine, and a roof in the training environment. It is preferable to notify the user of a good environment training menu reflecting the information which has been collected by the sensor S while the user is performing the training (second training) in the poor training environment.

In the embodiments of the invention, the information detected by the sensor S and the information obtained by the training machine M are sent to the training support apparatus 4 through the user terminal 2. But it is possible for the sensor S, the training machine M and other apparatus to send the information directly to the training support apparatus 4 not through the user terminal 2.

Further, in the embodiments of the invention, the exercise information of the “second training” is collected and the menu for the “first training” reflecting the collected exercise information is created, and then the created menu is notified to the user. On the contrary, it is possible to collect the exercise information of the “second training” and to create the menu for the “first training” reflecting the collected exercise information, and then to notify the created menu to the user.

Although specific embodiments of the invention have been described in the foregoing detailed description, it will be understood that the invention is not limited to the particular embodiments described herein, but modifications and rearrangements may be made to the disclosed embodiments while remaining within the scope of the invention as defined by the following claims. It is intended to include all such modifications and rearrangements in the following claims and their equivalents.—

What is claimed is:

1. A training support apparatus comprising:
a memory in which a first data table and a second data table are stored, wherein, in the first data table, calorie amounts consumed while a user performs respective gym trainings in a training gym are registered, and, in the second data table, calorie amounts consumed while the user performs respective self trainings are registered, the self trainings being different from the gym trainings;
a portable terminal which is used for exchanging data with a training machine provided in the training gym; and
a processor which executes a process,
wherein the process comprises:
a receiving process for receiving a training menu which the user is to perform, prior to the user's performing the gym training and the self training;
a self training support process for displaying a self training menu contained in the training menu received in the receiving process on a display device of the portable terminal assigned to the user, and for obtaining an exercise volume and physical information of the user from a sensor attached to the user, through the portable terminal, while the user performs the self training; and
a gym training support process for amending the gym training menu contained in the training menu received in the receiving process based on the exercise volume and the physical information of the user obtained in the self training support process, and for displaying the amended gym training menu on the display device of the portable terminal assigned to the user while the user performs the gym training.

2. The training support apparatus according to claim 1, wherein:
in the gym training support process, a total calorie amount is calculated, of a calorie amount derived from the exercise volume and the physical information of the user obtained in the self training support process and a calorie amount to be consumed while the user performs the amended gym training menu displayed on the display device of the portable terminal assigned to the user, and the gym training menu contained in the training menu received in the receiving process is amended such that the calculated total calorie amount will approach a calorie amount to be consumed, derived from the training menu received in the receiving process, and then the amended gym training menu is displayed.

3. The training support apparatus according to claim 1, wherein the gym training support process sets into the amended gym training menu the training machine to be used while the user performs the gym training.

4. The training support apparatus according to claim 1, wherein the gym training support process obtains an exercise volume of the user from a sensor built in the training machine used while the user performs the gym training.

5. The training support apparatus according to claim 4, wherein, in the self-training support process, the self training menu contained in the training menu received in the receiving process is amended based on the exercise volume of the user obtained in the gym training support process, and the amended self training menu is displayed on the display device of the portable terminal assigned to the user.

6. A training support method for a training support system comprising a memory and a portable terminal used for exchanging data with a training machine provided in a training gym, wherein the memory stores a first data table and a second data table, wherein, in the first data table, calorie amounts consumed while a user performs respective gym trainings in a training gym are registered, and, in the second data table, calorie amounts consumed while the user performs respective self trainings are registered, the self trainings being different from the gym trainings, the method comprising the steps of:
a training menu receiving step of receiving, by the portable terminal, a training menu which the user is to perform, prior to the user's performing the gym training and the self training;
a self training support step of displaying a self training menu contained in the training menu received at the training menu receiving step on a display device of the portable terminal assigned to the user, and of obtaining an exercise volume and physical information of the user from a sensor attached on the user through the portable terminal, while the user performs the self training; and
a gym training support step of amending the gym training menu contained in the training menu received at the training menu receiving step based on the exercise volume and the physical information of the user obtained at the self training support step, and of displaying the amended gym training menu on the display device of the portable terminal assigned to the user, while the user performs the gym training.

7. The training support method according to claim 6, wherein:
at the gym training support step, a total calorie amount is calculated, of a calorie amount derived from the exercise volume and the physical information of the user obtained at the self training support step and a calorie amount to be consumed while the user performs the amended gym training menu displayed on the display device of the portable terminal assigned to the user, and the gym training menu contained in the training menu received at the training menu receiving step is amended such that the calculated total calorie amount will approach a calorie amount to be consumed, derived from the training menu received at the training menu receiving step, and then the amended gym training menu is displayed.

8. The training support method according to claim 6, wherein, at the gym training support step, the training machine to be used while the user performs the gym training is set into the amended gym training menu.

9. The training support method according to claim 6, wherein, at the gym training support step, an exercise volume of the user is obtained from a sensor built in the training machine used while the user performs the gym training.

10. The training support method according to claim 9, wherein, at the self-training support step, the self training menu contained in the training menu received in the training menu receiving step is amended based on the exercise volume of the user obtained at the gym training support step, and the amended self training menu is displayed on the display device of the portable terminal assigned to the user.
